(19) United States
(12) Patent Application Publication
(10) Pub. No.: US 2021/0259781 A1
(43) Pub. Date: Aug. 26, 2021

(54) FORCE BASED DIGITIZATION FOR BONE REGISTRATION

(71) Applicant: THINK SURGICAL, INC., Fremont, CA (US)

(72) Inventors: Micah Forstein, Fremont, CA (US); Joel Zuhars, Fremont, CA (US); Eustache Felenc, Fremont, CA (US)

(73) Assignee: THINK SURGICAL, INC., Fremont, CA (US)

(21) Appl. No.: 17/182,417

(22) Filed: Feb. 23, 2021

Related U.S. Application Data
(60) Provisional application No. 62/981,746, filed on Feb. 26, 2020.

(51) Int. Cl. A61B 34/20; A61B 5/00; A61B 6/00; A61B 90/50; A61B 34/30; A61B 17/56; G16H 40/67; G16H 30/40; G16H 50/70; G16H 20/40; G16H 50/50

(52) U.S. Cl. CPC A61B 34/20; A61B 6/032; A61B 5/4887; A61B 6/505; A61B 5/7203; A61B 5/7246; A61B 90/50; A61B 34/30; A61B 5/742; A61B 17/56; G16H 40/67; G16H 30/40; G16H 50/70; G16H 20/40; G16H 50/50; A61B 5/0053

(57) ABSTRACT

A method and system is provided for registering the position and orientation (POSE) of a bone, where only data points that rest on the cortex of the bone are used to establish data points for determining the bone's POSE during a surgical procedure. The method collects the contact force and only collects a data point upon the removal at a specific threshold, which allows a digitizer to pass through the cartilage or soft tissue prior to the condition which defines when a data collection switch is closed. The collection of points is more consistent since the threshold value is normalized to hounds-field units of computed tomography (CT) data used for segmentation. The method utilizes a load cell to define a selection of a point based upon the release of what the point load applied is, as well as normalizing the activation threshold to the CT data of the bone.

### CROSS REFERENCE TO RELATED APPLICATIONS

This application claims priority benefit of U.S. Provisional Application Ser. No. 62/981,746 filed 26 Feb. 2020, the contents of which are hereby incorporated by reference.

### TECHNICAL FIELD

The present invention generally relates to computer-assisted surgery, and in particular to systems and methods for registering a bone during computer-assisted surgery.

### BACKGROUND

Throughout a lifetime, bones and joints become damaged and worn through normal use, disease, and traumatic events. Arthritis is a leading cause of joint damage, which can cause cartilage degradation, pain, swelling, stiffness, and bone loss overtime. If the pain associated with the dysfunctional joint is not alleviated by less-invasive therapies, the joint may need to be replaced with a procedure called total joint arthroplasty (TJR). TJR is an orthopedic surgical procedure in which the typically worn articular surfaces of the joint are replaced with prosthetic components, or implants. TJR typically requires the removal of the articular cartilage of the joint including a varying amount of bone. This cartilage and bone are then replaced with synthetic implants, typically formed of biocompatible metals, plastics, or composites to form the new synthetic joint surfaces.

The accurate placement and alignment of the implants on the bone is a major factor in determining the success of a TJR procedure. Even a slight misalignment of an implant may result in poor wear characteristics, reduced functionality, poor clinical outcomes, decreased implant longevity, or a combination of these outcomes. Therefore, several TJR procedures are now performed with computer-assistance, and even more advanced procedures utilize robotic surgical systems. One such robotic surgical system is the TSOLUTION ONE® Surgical System (THINK Surgical, Inc., Fremont, Calif.), which aids in the planning and execution of total hip arthroplasty (THA) and total knee arthroplasty (TKA). The TSOLUTION ONE® Surgical System includes: a pre-operative planning software program to generate a surgical plan using an image data set and/or 3-D models of the patient's bone, computer-aided design (CAD) models of several implants; and an autonomous surgical robot that precisely mills the bone to receive an implant according to the surgical plan.

Conventional interactive pre-operative planning software generates a three dimensional (3-D) model of the patient's bony anatomy from a computed tomography (CT) or magnetic resonance imaging (MRI) image dataset of the patient. A set of 3-D computer aided design (CAD) models of the manufacturer's prosthesis are pre-loaded in the software that allows the user to place the components of a desired prosthesis to the 3-D model of the bony anatomy to designate the best fit, position and orientation of the prosthesis to the bone. The surgical plan data may include a volume of tissue for modification that is defined relative to the anatomy such as a set of points in a cut-file to remove bone, or a set of virtual boundaries to sculpt the bone. The user can then save the pre-operative planning data to an electronic medium that is loaded and read by a surgical device to assist the surgeon intra-operatively in executing the plan.

In order for a computer-assisted surgical system to accurately prepare a bone, the bone needs to be registered to the surgical system. Registration determines the spatial position and orientation (POSE) of the bone relative to the coordinates of the surgical plan and/or surgical system.

Several registration procedures are known in the art, illustratively including pin-based, point-to-point, point-to-surface, laser scanning, image-free, and image registration, as described in U.S. Pat. Nos. 5,951,475, 6,033,415, 8,287,522, and 8,010,177. The most commonly used registration procedure relies on the manual collection of several points (i.e., point-to-point, point-to-surface) on the bone using a tracked digitizer probe where the surgeon is prompted to collect several points on the bone that are readily mapped to corresponding points or surfaces on a representation of the bone (e.g., a 3-D bone model). The points collected from the surface of a bone with the digitizer may be matched using iterative closest point (ICP) algorithms to generate a transformation matrix. The transformation matrix provides the correspondence between the position of the bone in an operating room (OR) with the bone model to permit the surgical device to execute the plan. The bone model is typically generated using CT data where the bone is segmented from the CT data. Therefore, the bone model is a good representation of the outside/cortex of the bone.

As described in U.S. Pat. No. 6,033,415 a probe is contacted against a bony surface at a plurality of locations to recognize or register the location of the bone and to digitize the surface of the bone. Once the surface of the bone has been digitized, the acquired data points can be compared against a pre-acquired model of the bone so as to identify the position of the bone in space. With the method of U.S. Pat. No. 6,033,415; the probe can digitize an exposed bony surface, or it can digitize the bony surface by puncturing the skin and contacting the bone. In practice, puncturing of the skin is desirable in some cases so as to reduce the size of the incision made in the patient; but also, it can be challenging to accurately digitize the bony surface through puncturing of the skin and tissue because the “view” is very limited and the tactile feedback is often compromised by the skin, muscle and periosteum. Furthermore, a problem may arise during the bone digitizing process in an OR since the bone is covered in soft tissues, it is possible that the surgeon does not pierce through the soft tissue, such as cartilage to actually make contact with the cortex/outer surface of the bone. As a result of these complications, collected points are often recorded a small distance beyond the surface of the bone thereby complicationg matching with a corresponding point on the 3-D bone model.

U.S. Pat. No. 8,615,286, incorporated herein in its entirety by reference, discloses a probe designed to make mechanical contact with a bony surface, where the probe is thin and long like a needle so that the probe can be introduced through the soft tissue until it contacts the bony surface. As the spatial location of the probe recognizes the location of the probe tip such that when the probe is in contact with the bone, that location will be digitized and processed by the computer-aided surgery system. For the recognition of the probe location, a mechanical arm type digitizer, an infra-red (IR) marker tracking camera, a magnetic tracker, or any other appropriate method and/or apparatus can be used. For detection of probe contact with the bony surface, a force sensor can detect the resistance of the material encountered by the probe, a piezoelectric sensor can measure the natural frequency of the probe, an ultrasound sensor can measure the material property at the tip of the probe, or any other appropriate method and/or apparatus can be used. The contact detector discriminates between (i) the force encountered by the tip of the needle when the tip of the needle is engaging soft tissue, and (ii) the force encountered by the tip of the needle when the tip of the needle is engaging bone.

FIGS. 1-4 show a series of prior art schematic diagrams of a probe that acts as a percutaneous bone detector 110 designed to make mechanical contact with a bony surface. FIG. 1 shows a prior art measurement system 100 showing the probe 110 making contact with a bone B of a patient lying in a supine position. The system 100 further includes a surgical robot 102, a computer controller 104, a display 106, and user input/output peripherals (e.g., mouse, pendent, keyboard, foot pedal, etc.). In order to measure the force acting on the needle 112 while knowing the location of the needle tip 114, a force sensor 116 and tracking device 122 with encoders 124 provide location and orientation information to a computer 104. The computer 104 is also used to acquire and process the force and distance data. With the force sensor 116 attached to the needle probe 110, the interface between two layers of tissue with different degrees of hardness can be distinguished via the rate of change of the force that the probe 110 encounters. More particularly, when a piercing probe 110 is pushed through tissue, it encounters varying resistance as it goes through different types of tissue. The probe 110 acting as the percutaneous bone detector utilizes this varying resistance to distinguish between soft tissue (i.e., fat and muscle) and hard tissue (i.e., bone). The piecing needle 112 portion of the probe 110 is a large diameter steel needle. A load cell that forms a force sensor 116 is attached to the probe base so that the resistant force is directly sensed by the force sensor 116. It is appreciated that the force sensor may take the form of a load cell, strain gauge, or a pressure sensor. It is further appreciated that the force sensor may also be located at the tip 114 of the probe needle 112, or other locations to measure forces on the tip 114. The distance traveled by the needle tip 114 is tracked by a set of encoders 128 on an arm 130. Thus, the relationship between the force and distance can be measured.

It is appreciated that signal processing, for example to reduce noise, is performed so as to eliminate randomness due to “looseness” of the setup. The derivative of the force with respect to distance of the probe tip is calculated. A threshold is determined heuristically for the soft tissue-bone tissue interface. Different bone types, different sections of the bone, the age of the patient, and health of the patient all have an effect on the calculated threshold.

FIG. 2 shows a prior art probe needle 112 as it pierces through the skin and penetrates across different tissue layers (L1—cartilage, L2—cortical bone, L3—trabecular bone) of tissues and prior to the tip 114 reaching the bone B of a limb. The setup 100 measures the resistance acting on the needle and the travel (distance) of the needle. The computer 104 digitizes the two measurements and processes them to obtain the bone surface information.

FIG. 3 is a more detailed view of the prior art probe tracking system 100. The probe 110 is installed on a robot arm 130 with revolute joints 128. The base of the robot arm is attached on a fixture. A load cell 116 for force measurement that resides in a handle 118 is attached to the needle probe 112. A computer 104 receives signals from the rotary encoders in the revolute joints 128 to compute the position of the probe tip 114.

FIG. 4 shows the internal structure of the prior art probe 110. The handle 118 contains a load cell/force sensor 116 that senses force translated from the needle/digitizer probe 112 when piercing tissue in a subject. When assembling the probe 110, the needle is inserted first into the front piece of the handle 118. The back piece of the handle 118 is attached to the front piece with the load cell 116 therein with the loading area facing the needle base. Washers 120 are placed in the assembly to eliminate free play between the load cell 116 and the needle 112. A tracking array 122 is fixedly attached to the handle 118 for tracking the position and orientation of the probe 110. The tracking array 122 has a set of fiducials markers 124. In the embodiment shown, a light emitting diode (LED) data transmitter 126 communicates measured force data to the computer 104. In other embodiments a data cable may be used to transfer force data to the computer 104. It is noted that a piezoelectric force sensor can replace a load cell (which is a strain gauge) in this apparatus.

While there have been advancements in the establishment of the position and orientation of bones during surgical procedures, the process still requires the selection of multiple points at which the probing digitizer tip rests on the outside of the bone cortex. As a result, the tip can easily be occluded by cartilage or soft tissue while performing a surgical procedure using a navigation system or surgical robot. Thus, while surgeons are required to penetrate through the cartilage and touch the underlying bone before picking a point for registration, failure to transit through the cartilage to the underlying bone still occurs and results in a failed registration procedure or worse, successful registration but misplaced implants. Therefore, there continues to be a need for a method and a device for performing such a method to ensure the proper registration of bones in a surgical procedure. There further exists a need to inhibit human error in contacting cortical bone for registration measures associated with a surgical procedure.

### SUMMARY OF THE INVENTION

A method is provided for determining the position and orientation of a bone of a patient. The method includes directing a digitizer tip onto tissue overlying the bone to collect a surface point location. The forces are exerted on the digitizer tip are monitored. When a force exerted on the digitizer tip by contact with the bone exceeds a predetermined threshold force is determined. When the predetermined threshold force is exceeded, the digitizer tip is removed by moving the digitizer tip away from the bone to reduce the force exerted on the digitizer tip. A location of the digitizer tip at an instant when the force is equal to, or less than the threshold force is recorded. The method continues with repeatedly directing the digitizer tip to different locations on the bone to collect additional surface point locations until a desired number of surface points have been recorded to register the bone. The threshold force can be based on premeasured bone hardness data obtained from a computed tomography (CT) scan of the bone that is used to determine expected forces exerted on the digitizer tip when the digitizer tip contacts cortical bone.

Another method is provided for determining the position and orientation of a bone of a patient. The method includes obtaining anatomy imaging data, where the anatomy imaging data includes voxels, where each voxel has an estimated tissue density value. The estimated tissue density values are correlated to an expected force value, where the expected force value is an expected measurement of force on a digitizer tip while digitizing a surface point. The method further includes a digitizer tip directed onto tissue overlying the bone to collect a surface point location. The forces exerted on the digitizer tip are monitored. The forces on the digitizer tip and the position of the digitizer tip are recorded while digitizing the surface point to generate a series of points. The series of points each have a positional depth with an associated recorded force value or range of values. Two or more points in the series of points are correlated with corresponding points in the anatomy imaging data based at least partially on a similarity between the expected force values and the recorded force values, where each point correlation represents a layer of tissue. A transformation matrix is calculated for a tissue layer using point correlation from at least some of the surface points digitized for the tissue layer. At least one calculated transformation matrix is combined with additional matrix data to complete registration of the bone. The digitizer tip is repeatedly directed to different locations on the bone to collect additional surface point locations until a desired number of surface points have been recorded to establish the position and orientation of the bone. A transformation can be calculated for each tissue layer using an iterative closest point (ICP).

Still another method is provided for determining the position and orientation of a bone of a patient. The method includes obtaining imaging data from a CT scan of the bone. The method further includes directing a digitizer tip onto tissue overlying the bone to collect a surface point location, monitoring forces exerted on the digitizer tip, and recording both the forces on the digitizer tip and the position of the digitizer tip while digitizing the surface point to generate a series of points, where each point in the series of points has a positional depth with an associated recorded force value or range of values. Two or more points in the series of points are correlated with corresponding points in the anatomy imaging data based at least partially on a similarity between an expected force threshold and the recorded force values, where each point correlation represents a tissue layer overlying the bone. At least one transformation matrix is calculated for the tissue layer using each point correlation from at least some of the surface points surface points digitized for that given tissue layer overlying the bone. The at least one transformation matrix is combined with additional matrix data to complete registration of the bone. The digitizer tip is repeatedly directed to different locations on the bone to collect additional surface point locations until a required number of surface points have been recorded to establish the position and orientation of the bone. A transformation can be calculated for the tissue layer using iterative closest point (ICP) algorithms.

A computer-assisted surgical system is provided that includes a percutaneous bone detector having a digitizer tip to collect a set of surface point locations on a bone, a tracking system, a surgical robot with an end effector, and one or more computers with software. The one or more computers receive electric signals from the tracking system which tracks the position of the digitizer tip and records forces exerted on the digitizer tip. The system further includes a display to display the output from the one or more computers in real-time. The one more computers include software to execute instructions to perform at least one of the following: (a) record a position of the digitizer tip when a force on the digitizer tip is equal to or less than a threshold force; or (b) calculate at least one transformation matrix representative of a tissue layers and combine the at least one transformation matrix with additional matrix data to register the bone indicative of the position and orientation of the bone.

### BRIEF DESCRIPTION OF THE DRAWINGS

The present invention is further detailed with respect to the following drawings that are intended to show certain aspects of the present of invention, but should not be construed as a limit on the practice of the invention, wherein:

FIG. 1 shows a prior art schematic diagram of a percutaneous bone detector with the patient lying down supine;

FIG. 2 shows a prior art illustration of the percutaneous bone detector of FIG. 1 penetrating soft tissue centrally toward a bone;

FIG. 3 shows a prior art example of a probe tracking system with the percutaneous bone detector of FIG. 1 having a load cell installed on a robot arm that has revolute joints that is attached to a fixture, and where a computer receives rotary encoder signals from the revolute joints to compute the probe tip position;

FIG. 4 is a detailed prior art depiction of an optically tracked percutaneous bone detector having a force sensor with tracking array;

FIG. 5 illustrates a method to collect points during registration based on a sensed removal of a threshold force in accordance with an embodiment of the invention;

FIG. 6 illustrates a method to estimate the threshold forces used in the method of FIG. 5 (Block 208) using the bone imaging data in accordance with an embodiment of the invention;

FIG. 7 illustrates a method to register a bone by calculating multiple transformations for different layers of tissue in accordance with an embodiment of the invention;

FIG. 8 illustrates a method to register a bone by calculating multiple transformations for different tissue layers using force thresholds in accordance with an embodiment of the invention;

FIG. 9 depicts computed tomography (CT) imaging data of a bone surrounded by other tissues in the form of a grid populated with Hounsfield Units (HU) in accordance with an embodiment of the invention;

FIG. 10 depicts a graph of the expected and measured forces sensed by the force sensor as a function of tissue depth while collecting a surface point in accordance with embodiments of the invention;

FIGS. 11A and 11B depict the matching of points for different tissue layers between a bone model and an actual bone, respectively to calculate multiple transformations in accordance with embodiments of the invention; and

FIG. 12 depicts a surgical system in the context of an operating room (OR) with a surgical robot for implementing the method of FIGS. 5-8 in accordance with embodiments of the invention.

### DETAILED DESCRIPTION

The present invention has utility as a system and method for improving the accuracy of registering the position and orientation (POSE) of a bone during a surgical procedure. The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention.—Hence, the following specification is intended to illustrate some particular embodiments of the invention, and not to exhaustively specify all permutations, combinations, and variations thereof.

All publications, patent applications, patents and other references mentioned herein are incorporated by reference in their entirety.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

As used herein, the term “digitizer” refers to a measuring device capable of measuring physical coordinates in three-dimensional space. For example, the ‘digitizer’ may be: a “mechanical digitizer” having passive links and joints, such as the high-resolution electro-mechanical sensor arm described in U.S. Pat. No. 6,033,415; a non-mechanically tracked digitizer probe (e.g., optically tracked, electromagnetically tracked, acoustically tracked, and equivalents thereof) as described for example in U.S. Pat. No. 7,043,961; a digitizer probe as described in U.S. Pat. No. 8,615,286; or an end-effector of a robotic device.

As used herein, the term “digitizing” refers to the collecting, measuring, and/or recording of physical points in space with a digitizer.

As used herein, the term “pre-operative bone data” refers to bone data used to pre-operatively plan a procedure before making modifications to the actual bone. The pre-operative bone data may include one or more of the following. An image data set of a bone (e.g., computed tomography, magnetic resonance imaging, ultrasound, x-ray, laser scan), a virtual generic bone model, a physical bone model, a virtual patient-specific bone model generated from an image data set of a bone, or a set of data collected directly on a bone intra-operatively commonly used with imageless computer-assist devices.

Also described herein are “computer-assisted surgical systems.” A computer assisted surgical system refers to any system requiring a computer to aid in a surgical procedure. Examples of computer-assisted surgical systems include 1-N degree of freedom hand-held surgical systems, tracking systems, tracked passive instruments, active or semi-active hand-held surgical devices and systems, autonomous serial-chain manipulator systems, haptic serial chain manipulator systems, parallel robotic systems, or master-slave robotic systems, as described in U.S. Pat. Nos. 5,086,401; 7,206,626; 8,876,830; 8,961,536; and 9,707,043; and PCT Publication WO2016049180. In particular inventive embodiments, the surgical system is a robotic surgical system as described below. In particular inventive embodiments, the surgical system is a 2-DOF articulating device as described in U.S. Patent Publication 2018/0344409. The surgical system may provide autonomous, semi-autonomous, or haptic control and any combinations thereof. In addition, a user may manually maneuver a tool attached to the surgical system while the system provides at least one of power, active, or haptic control to the tool.

As used herein, the term “registration” refers to the determination of the POSE and/or coordinate transformation between two or more objects or coordinate systems such as a computer-assist device, a bone, pre-operative bone data, surgical planning data (i.e., an implant model, cut-file, virtual boundaries, virtual planes, cutting parameters associated with or defined relative to the pre-operative bone data), and any external landmarks (e.g., a tracking marker array) associated with the bone, if such landmarks exist. Methods of registration known in the art are described in U.S. Pat. Nos. 6,033,415, 8,010,177, and 8,287,522.

Also, referenced herein is a surgical plan. For context, a surgical plan is created, either pre-operatively or intra-operatively, by a user using planning software. The planning software may be used to plan the position for an implant relative to pre-operative bone data. For example, the planning software may be used to generate three-dimensional (3-D) models of the patient's bony anatomy from a computed tomography (CT), magnetic resonance imaging (MRI), x-ray, ultrasound image data set, or from a set of points collected on the bone intra-operatively. A set of 3-D computer aided design (CAD) models of the manufacturer's prosthesis are pre-loaded in the software that allows the user to place the components of a desired prosthesis to the 3-D model of the bony anatomy to designate the best fit, position, and orientation of the implant to the bone.

As used herein, the term “real-time” refers to the processing of input data within milliseconds such that calculated values are available within 10 seconds of computational initiation.

Also used herein is the term “optical communication” which refers to wireless data transfer via infrared or visible light that are described in U.S. Pat. No. 10,507,063 with respect to visible light and assigned to the assignee of the present application and incorporated by reference herein in its entirety.

Embodiments of the invention provide for improved registration of bones to a surgical robot when using a contact probe to determine registration points on the surface of a bone. In a surgical procedure selection of a point using a digitizer requires that the point rest on the outside of the cortex of the bone which can easily be occluded by cartilage and soft tissue overlying the bone while performing a surgical procedure using a navigation system or surgical robot. Surgeons are required to poke through the cartilage and touch the bone behind it before picking a point for registration, since failure to do so results in errors when establishing data points for determining the position and orientation (POSE) of a bone during a surgical procedure. However, some surgeons fail poking through the cartilage (for example, because they forget they have to) which can result in failed registration procedure or worse, successful registration procedures but misplaced implants.

In a specific inventive embodiment, a method is provided for ensuring that only data points that rest on the cortex of the bone are used to establish data points for determining the position and orientation (POSE) of a bone during a surgical procedure. The inventive method collects the contact force and only collects a data point upon the removal at a specific threshold, which will allow the digitizer to pass through the cartilage or soft tissue prior to the condition which defines when a data collection switch is closed. It is appreciated that the present invention also is operative to collect a data point for bare bone portions as well as portions of bone having overlying soft tissue thereon. In addition, this process allows for the collection of points more consistently provided the threshold value is normalized to the hounds-field unit of the CT data used for segmentation. The inventive method utilizes a load cell to define a selection of a point based upon the release of what the point load applied is, as well as normalizing the activation threshold to the CT data of the bone.

While the present invention is detailed with respect to digitizing a bone, if should be appreciated that the inventive steps are readily duplicated on a second bone. In many instances, the second bone in combination with the bone define an anatomical joint, or portion thereof.

In a specific inventive embodiment, a method is provided that utilizes the fact that cartilage and bone have very different hardness to automatically collect a surface point with a digitizer. In order to avoid human error or judgement to influence the selection of bone surface data points, a digitizer is designed to not require a conventional manual input such as a switch or button to collect a point, and instead, the present invention applies sufficient force to penetrate through the cartilage and trigger a surface point collection when the bone is reached. In this inventive embodiment, a gauge in the digitizer is calibrated such that collection of a data point will only trigger a point collection signal when applied pressure is above a predetermined threshold. A gauge operative herein includes a strain gauge, laser vibrometer, inductive, a capacitive transducer, or any other force or pressure sensor. The method relies on the material property differences between cartilage and soft tissue overlying the bone, as compared to the underlying bone is so different that calibration of the gauge is not required to be overly sensitive. Furthermore, the bone material properties detected by the gauge, such as hardness, strongly correlate to CT scan Hounsfield values. Hounsfield units (HU) are a dimensionless unit universally used in computed tomography (CT) scanning to express CT numbers in a standardized and convenient form. Hounsfield units are obtained from a linear transformation of the measured attenuation coefficients. The Hounsfield unit (HU) is a relative quantitative measurement of radio density used by radiologists in the interpretation of computed tomography (CT) images. The absorption/attenuation coefficient of radiation within a tissue is used during CT reconstruction to produce a grayscale image. It is appreciated that false color is readily applied to such an image to overlie additional data such as the force-based data obtained by the present invention.

In a further specific inventive embodiment, a method is provided that utilizes a point force profile as compared to a CT density profile of a bone to improve registration accuracy. Small errors in the registration can occur due to noise with the measuring equipment, segmentation errors while generating the bone model, and human errors. An inventive method also attempts to improve registration by accounting for variable attributes between patients as to factors such as tissue quality, bone density, or a combination thereof. A series of quantized force profiles for a given CT scan of bone densities are used to create a registration from the bone model to the transform of data for the computer aided surgical procedure. This inventive method embodiment takes into consideration the pliability of the bone and soft tissue overlying the bone as a function of the bone density to create multiple transforms. Each created transform is mapped to a combination of bone to robot transforms that are weighted by a segmentation fit. It is appreciated that the transform to a frame of reference associated with a robot of a surgical system facilitates more accurate bone removal in the course of a surgical procedure or anatomy modeling. Anatomy modeling is typically used to design a custom implant. Some inventive embodiments utilize a load cell on the digitizer tip or handle and acquisition of the position profile is as a function of the force applied for each digitized point location on the bone. Each point collected then generates a series of points at different depths as a function of forces, these points are then matched to other points at that same force to create a digitized bone model for each force level. The resulting force data for surface points on the bone or other tissue layers can be used to populate a transformation matrix using an iterative closest point (ICP) algorithm. Once the ICP algorithm is applied, each transformation matrix is weighted, based upon the point approximation to the segmented bone model, and each weighted set is then combined statistically to reduce the noise and variability of the transform between the bone and the robot.

For example, if there is a set of layered materials that independent of force may only be pierced with a specific diameter of needle. Then in some inventive embodiments, one could measure the location of the underlying surface, for example that of hard cortical bone, without measuring overlying surfaces and by changing the diameter of the needle, measure the profile of these overlying layers. In a similar manner, the same principle may applied to a force profile that is if data collection is tied to 4 Newtons (N) of applied force, effectively acting as a force switch, then one would only get a result if the digitizer is pushed with 4 pounds and so the surface created would be a 4 N surface. If there are multiple switch levels (e.g., 2 N, 4N, 6N) then one could obtain a surface that corresponds to each of those force levels. Then each force level may be correlated back to density of the CT through testing and one could effectively build a map of the different levels and then use all of those to register the bone location and even chart the bones quality. Because all of the forces are in the same position from a digitizer standpoint, one can use the multiple force models to correlate the registration position back to the CT scan data. It is appreciated that such a force topography mapping of a bone is also readily formed with a force gauge that measure dynamic values, as opposed to preset values as detailed above. It is appreciated that the force topography mapping may be visually represented as a color spectrum where different colors along a continuum represent force magnitudes. It is also appreciated that the force topography may be represented in a grayscale, where for example a darker shade is representative of a higher applied force which equates to a higher density of the CT and of hardness of the layer. It is further appreciated that a series of gradient lines where a denser grouping of lines is representative of a harder region may also be used as an indicator of applied forces for corresponding layers.

A force sensor is a sensor capable of converting force into a measurable electrical output and may operate in a hydraulic, pneumatic, piezoelectric, laser vibrometer, or capacitive manner. In particular embodiments, the force sensor is a strain gauge, which is a sensor whose resistance varies with applied force. A strain gauge converts force, pressure, tension, weight, etc., into a change in electrical resistance which can then be measured. When external forces are applied to a stationary object, stress and strain are the result. Stress is defined as the object's internal resisting forces, and strain is defined as the displacement and deformation that occur. Strain includes tensile and compressive strain, distinguished by a positive or negative sign. Thus, strain gauges can be used in the present invention to pick up expansion as well as contraction. The strain of a body is always caused by an external influence or an internal effect. Strain might be caused by forces, pressures, moments, heat, structural changes of the material and the like. If certain conditions are fulfilled, the amount or the value of the influencing quantity can be derived from the measured strain value. In experimental stress analysis this feature is widely used. Experimental stress analysis uses the strain values measured on the surface of a specimen, or structural part, to state the stress in the material and also to predict its safety and endurance. Special transducers can be designed for the measurement of forces or other derived quantities. These other properties that are illustratively measured include moments, pressures, accelerations, displacements, vibrations and others. A transducer generally contains a pressure sensitive diaphragm with strain gauges bonded thereto.

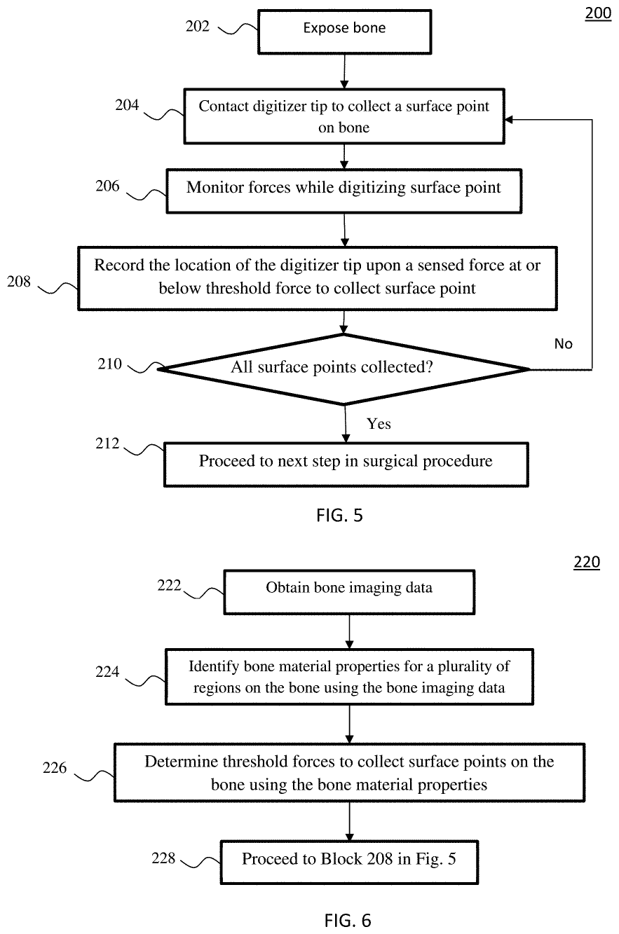

Referring now to the figures, FIG. 5 illustrates a flow diagram of an embodiment of an inventive method 200 that collects points that correspond to the position of the probe tip during registration based on the sensed removal of a threshold force. The method in some inventive embodiments begins with the surface of the bone being exposed (Block 202), while in other inventive embodiments, a digitizer tip pierces intact or partially resected tissue covering the bone. Regardless of the bone exposure, the digitizer tip is directed into bone contact to collect a surface point location on the bone (Block 204) while monitoring forces exerted on the digitizer tip (Block 206). When the force exerted on the digitizer tip exceeds the threshold value, the location of the digitizer tip is recorded at the threshold level as the force is removed by moving the digitizer tip away from the bone (Block 208). If additional surface points need to be recorded (Decision Block 210 is No), the digitizer tip is moved to a different location on the bone to collect a second surface point (Block 204). If the required number of surface points are recorded (Decision Block 210 is Yes), the process proceeds to the next step in the surgical procedure (Block 212). The next step of the surgical procedure may illustratively include calculating the transformation between the collected surface points and the imaging data. However, it should be appreciated that the calculation of the transformation may be performed in real-time as the surface points are being collected.

As shown in the inventive method flowchart of FIG. 5, as data is collected for a particular surface point on the bone, the force sensor measures and records the forces on the digitizer tip. Experimental bone hardness data is stored in the system to know what the expected force (or expected threshold force) will be exerted on the digitizer tip when it contacts cortical bone (e.g., 10 Newtons (N)). The forces on the digitizer tip should eventually reach the threshold force (e.g., 10 Newtons) indicating the digitizer tip is on cortical bone. However, it is appreciated that the measured forces could be much higher depending on how hard the surgeon is pushing the tip into contact with the bone. Therefore, the point isn't collected until the force sensor measures a force below the threshold (i.e., removal of the specified threshold or 10 Newton load); such as for example, as the surgeon begins pulling away from the bone, the measured forces will drop below 10N, at which time, the point is immediately collected that records the position of the probe tip upon removal of the threshold force.

FIG. 6 illustrates a flowchart of an embodiment of a method to estimate the threshold forces used in the method of FIG. 5 in (Block 208). Imaging data of the bone is obtained (Block 222) via a CT scan of the bone. Using the imaging data from the CT scan bone material properties for a plurality of regions of the bone are identified, such as bone densities, voids, and hardness (Block 224). Based on the identified bone properties in the plurality of bone regions threshold forces are determined for each of the plurality of bone regions (Block 226). Subsequently, the determined force thresholds are applied to record the position of the digitizer tip in Block 208 of the method of FIG. 5. The threshold forces may be normalized using the CT data, where the normalized threshold forces are used in the method of FIG. 5. The Hounsfield units in the CT data can be correlated to forces that are measured on the digitizer tip. For example, through experimental data, it may be expected that HU's in the range of 20-30 HUs will produce a measured force on the digitizer tip of 8 N. One may then go back into the CT data, and look at the HUs around the cortical bone for each point to be collected and specify the threshold forces that will be encountered. This provides the threshold forces to be used in the method of FIG. 5 (e.g., instead of 10 Newtons used in the above example, the value would then be adjusted to 8 Newtons in this example for any points (bone regions) having an HU around 20-30 HUs).

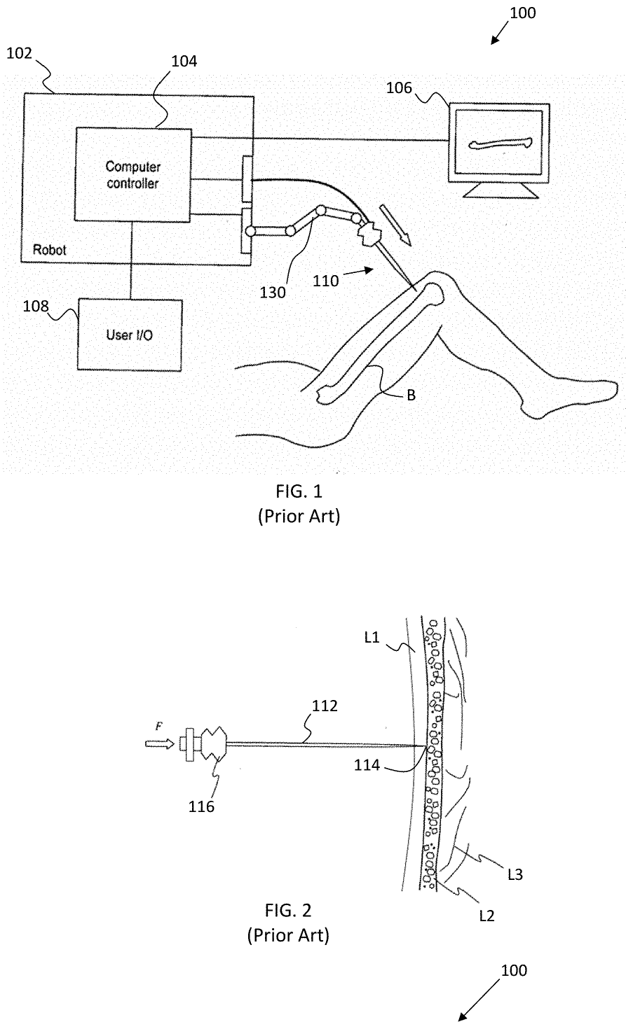

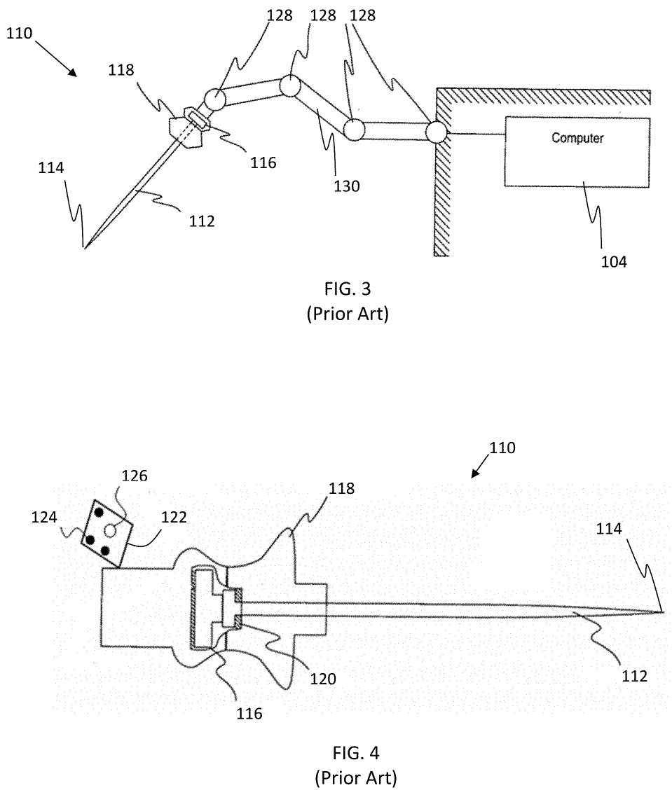

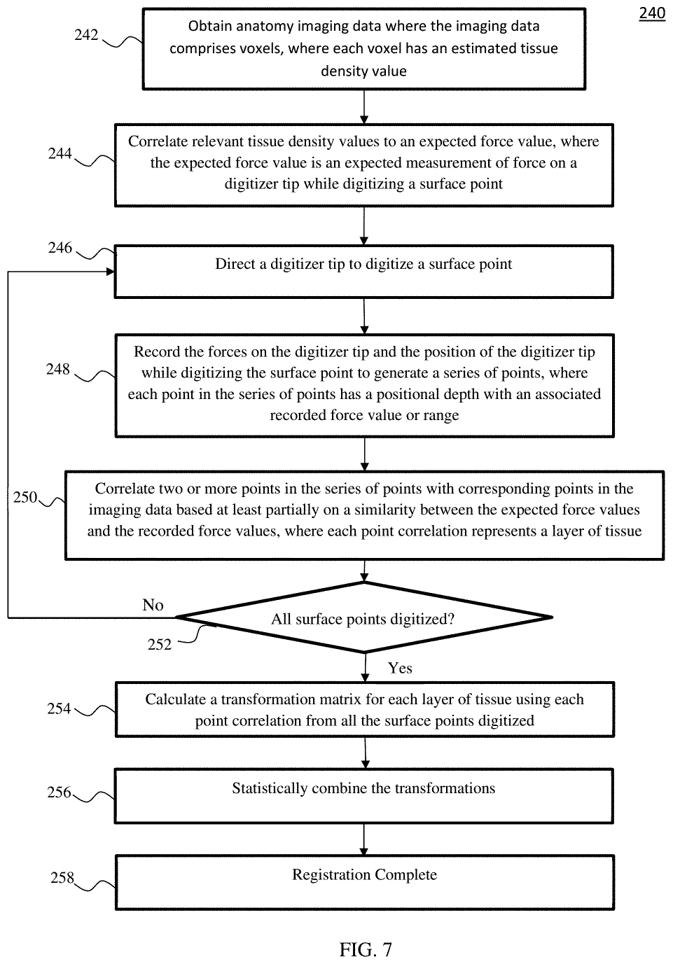

FIG. 7 illustrates a flowchart of an embodiment of a method 240 to register the bone by calculating multiple transformations for different layers of tissue overlying the bone. Anatomy imaging data is obtained where the imaging data includes voxels, where each voxel has an estimated tissue density value (Block 242). The tissue density values are correlated to an expected force value, where the expected force value is an expected measurement of force on a digitizer tip while digitizing a surface point (block 244). A digitizer tip is directed to digitize a surface point (Block 246), and the forces on the digitizer tip and the position of the digitizer tip are recorded while digitizing the surface point to generate a series of points, where each point in the series of points has a positional depth with an associated recorded force value or range (Block 248). Two or more points in the series of points are correlated with corresponding points in the imaging data based at least partially on a similarity between the expected force values and the recorded force values, where each point correlation represents a tissue layer (Block 250). A determination is made if the required number of surface points have been recorded to establish the position and orientation of the bone (Decision Block 252). If additional surface points need to be recorded (Decision Block 252 is No), the digitizer tip is moved to a different location on the bone to collect a surface point (Block 246). If the required number of surface points are recorded (Decision Block 252 is Yes), the process proceeds to calculate a transformation matrix for a tissue layer using each point correlation from all the surface points digitized (Block 254). The transformations from the transformation matrix are then statistically combined (Block 256) and the registration is completed (Block 258). It is appreciated that at least one transformation matrix generated according to the present invention can be combined with additional matrix data to complete the registration. Additional matrix data includes other transformation matrices generated by the present invention; conventional data generated by the prior art techniques such as those detailed with respect to FIGS. 1-4, ultrasonic positional data, or a combination thereof.

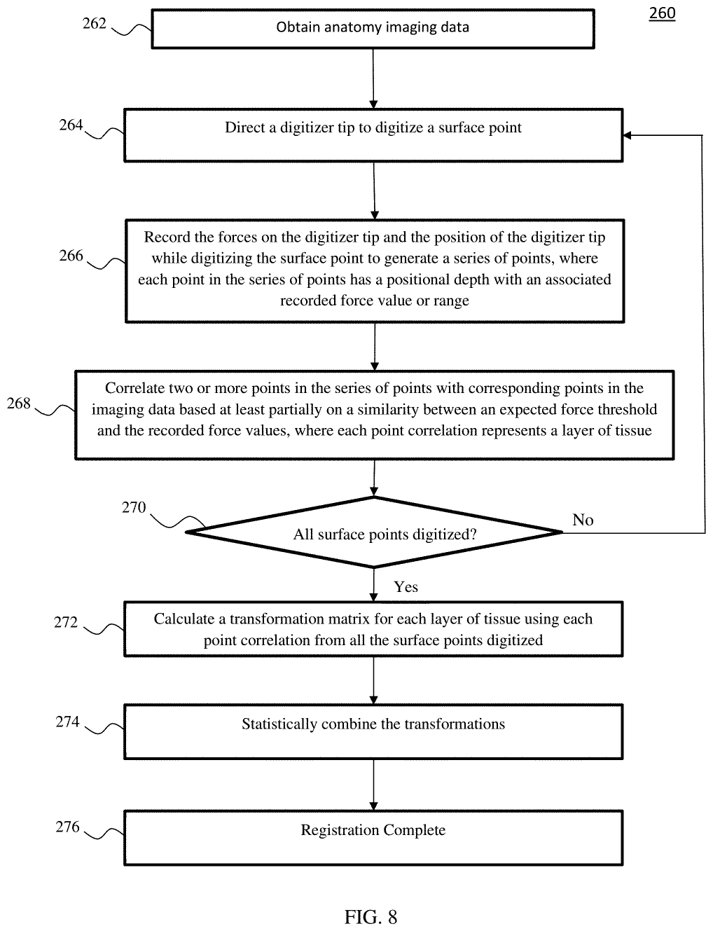

FIG. 8 illustrates a flowchart of an embodiment of a method 260 to register the bone by calculating multiple transformations for different tissue layers overlying the bone using force thresholds. Imaging data of the bone is obtained (Block 262) via a CT scan of the bone. A digitizer tip is directed to collect a surface point location on the bone (Block 264), while recording both forces exerted on the digitizer tip and the position of the digitizer tip while digitizing the surface point to generate a series of points, where each point in the series of points has a positional depth with an associated recorded force value or range (Block 266). The two or more points in the series of points are correlated with corresponding points in the imaging data based at least partially on a similarity between an expected force threshold and the recorded force values, where each point correlation represents a tissue layer (Block 268). A determination is made if the required number of surface points have been recorded to establish the position and orientation of the bone (Decision Block 270). If additional surface points need to be recorded (Decision Block 70 is No), the digitizer tip is moved to a different location on the bone to collect a surface point (Block 264). If the required number of surface points are recorded (Decision Block 270 is Yes), the process proceeds to calculate a transformation matrix for a tissue layer using each point correlation from all the surface points digitized (Block 272). It is appreciated that additional matrices are readily developed for other overlapping tissue layers. The transformations from the transformation matrix are then statistically combined (Block 274) and the registration is completed (Block 276). It is appreciated that at least one transformation matrix generated according to the present invention can be combined with additional matrix data to complete the registration. Additional matrix data includes other transformation matrices generated by the present invention; conventional data generated by the prior art techniques such as those detailed with respect to FIGS. 1-4, ultrasonic positional data, or a combination thereof.

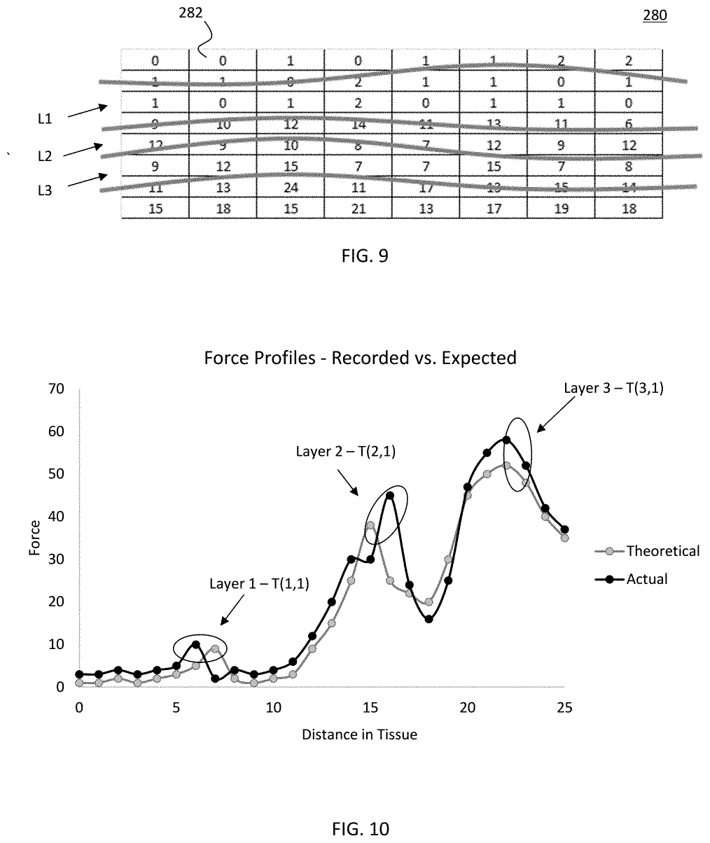

FIG. 9 depicts computed tomography (CT) imaging data of a bone surrounded by other tissues in the form of a grid 280 populated with Hounsfield Units (HU) in cells 282. Overlaid on the grid 280 are lines that divide the grid into layers (L1, L2, L3) of tissue overlying the bone found in a subject that a CT scan was performed on. For example, L1 may represent a first tissue layer overlying the bone in the image data with corresponding cells with HU values shown in that layer; L2 may represent a second tissue layer in the image data with corresponding cells with HU values shown in that layer; and L3 may represent a third tissue layer in the image data with corresponding cells with HU values shown in that layer. In the example shown, the bottom layer (L3) appears to be bone with the highest Hounsfield units.

Through experimental data (e.g., taking CT data from a cadaver with HU, digitizing the cadaver while measuring the forces on the digitizer tip, and correlating the measured forces back to the HUs; or from historical data of actual patients), the HU values can be correlated to an expected force measured on the digitizer tip. For example, an HU value between 12 and 15 likely results in a measured force on the digitizer tip of 6 Newtons. Thus, the HUs in the image data can be replaced by a force value or range.

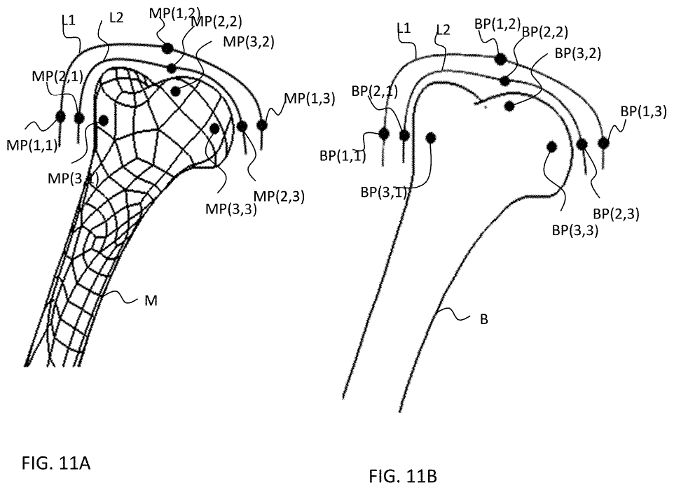

FIG. 10 depicts a graph of an example of predicted forces and measured forces sensed by the force sensor as a function of tissue depth while collecting a surface point. The layer transitions are circled in the graph. As can be seen the layers get harder as the probe needle nears the surface of the bone. The predicted forces may be estimated using the experimental/historical data and HUs in the imaging data as described above with reference to the description of FIG. 9. For example, the surface point to be collected might be the point represented on the last column, last row (18 HU) of FIG. 9. The HUs above this cell may be used to estimate the forces on the digitizer tip for the different tissue layers (e.g., 18 HU=12 N, 8 HU=8 N, 6 HU=4N . . .). Then while collecting the surface point on the bone, the force sensor measures the forces on the digitizer tip as a function of depth to create a series of points, where each point in the series of points has a positional depth with an associated recorded force. The points in the series of points are correlated/matched with corresponding points in the imaging data based at least partially on a similarity between the expected force values and the recorded force values, where each point correlation represents a layer of tissue overlying the bone. This is shown in the graph of FIG. 10, where the expected forces and the recorded forces are matched. The position of the digitizer tip is recorded for each force level, and therefore points or positions of the imaging data can be matched therewith based on the correlation/matching of the forces. It is noted that FIG. 10 is for the collection of a single surface point. The process is repeated for the collection of all the surface points. Then, a transform is calculated for each tissue layer (or correlation of points as a function of force or a force threshold). For example, in FIG. 10, a first tissue layer (Layer 1) is shown, where a transformation for layer 1 is calculated using the correspondence of points for that force or force threshold indicative of layer 1. The same goes for the second tissue layer (Layer 2), and the third tissue layer (Layer 3). This is better illustrated in FIGS. 11A and 11B.

FIGS. 11A and 11B depict the matching of points for different tissue layers between a bone model M and an actual bone B, respectively for calculation of multiple transformations to register the bone model to the actual bone B. As shown, there are two layers L1 and L2 that encompass the underlying bone (M, B). Surface points are identified bone model points MP and digitized points on actual bone BP. The pair of numbers in parenthesis next to each of the points correspond to transformation #/tissue layer, and surface point #. For example in FIG. 11B, BP(2,2) identifies the digitized point on tissue layer 2 that is surface point 2 with respect to the actual bone B.

The bone model of FIG. 11A illustratively has three surface points (MP(3,1), MP(3,2), and MP(3,3)) to be matched with three surface points on the actual bone (BP(3,1), BP(3,2), and BP(3,3)). A user may start the registration procedure by directing the digitizer tip to collect surface point BP(3,1). As the user collects the surface point, the force sensor measures the forces on the digitizer tip as it goes through the different tissue layers. The force sensor may record a force of 6 N at point BP(1,1), 8 N at point BP(2,1), and 10 N at point BP(3,1). From the imaging and experimental data as described in FIG. 9 and FIG. 10, the bone model or data associated with the bone model have points or positions with similar expected force values. For example, 6.2 N at point MP(1,1), 7.9 N at point MP(2,1), and 10 N at point MP(3,1). The points in the series of points for the collection of surface point BP(3,1) are correlated/matched with corresponding points in the imaging data based at least partially on a similarity between the expected force values and the recorded force values, where each point correlation represents a layer of tissue. That is, point BP(3,1) is correlated to point MP(3,1) based on the similarity in forces or a force threshold—bone layer, point BP(2,1) is correlated to point MP(2,1) based on the similarity in forces or a force threshold—layer 2 L2, and point BP(1,1) is correlated to point MP(1,1)—layer 1 L1 based on the similarity in forces or a force threshold. It should be appreciated that the collection of surface point BP(3,1) may be collected and correlated to point MP(3,1) using traditional point collection techniques (e.g., collecting a point using an input device when the digitizer tip is on the bone), or the technique described with reference to FIG. 5 and FIG. 6, rather than using the force correlation. This process is repeated for the collection of the other surface points BP(3,2) and BP(3,3). Once enough surface points are collected, a transformation can be calculated for each tissue layer using ICP algorithms. For example, to calculate the transformation of layer 1, the ICP algorithm matches points BP(1,1) with MP(1,1), BP(1,2) with MP(1,2), and BP(1,3) with MP(1,3). To calculate the transformation of layer 2, the ICP algorithm matches points BP(2,1) with MP(2,1), BP(2,2) with MP(2,2), and BP(2,3) with MP(2,3). The transformation for the bone surface points (BP(3,1), BP(3,2) and BP(3,3)) can be matched with their corresponding bone model points using the same method or traditional point collection techniques. This provides three transformation matrices, one for layer 1, one for layer 2, and one for the bone layer. Each transformation can then be weighted based upon the point approximation to the segmented bone model. For example, the transformation for layer 1 may be weighted less than the transformations for layer 2, and the bone layer, because these points are farthest from the bone and may not be as reliable. Each weighted transformation is then combined statistically to reduce the noise and variability of the transform between the bone and the robot.

It is appreciated that the surface points in the bone model as well as on the actual bone may be visually represented as a color spectrum where different colors along a continuum represent force magnitudes. It is also appreciated that the surface points may be represented in a grayscale, where for example a darker shade is representative of a higher applied force which equates to a higher density of the CT and of hardness of the layer at that specific surface point. It is further appreciated that a series of gradient lines where a denser grouping of lines is representative of a harder region may also be used as an indicator of applied forces for corresponding layers at each surface point.

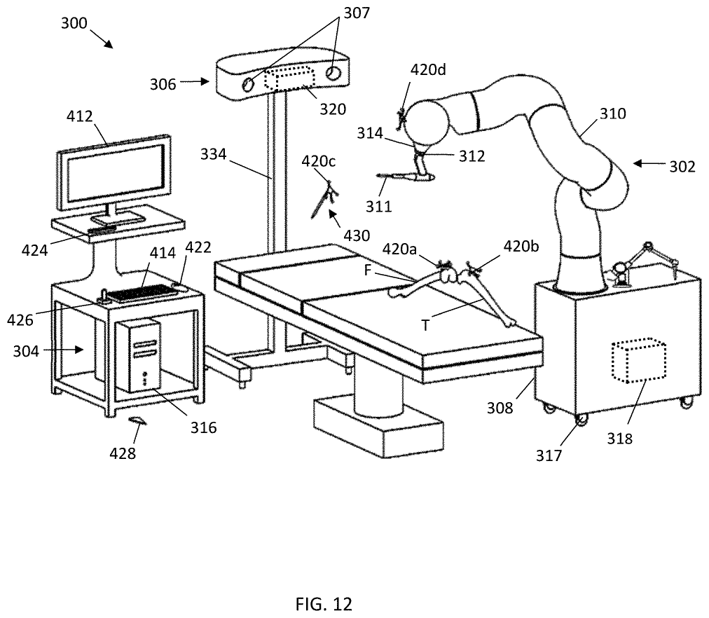

FIG. 12 depicts a surgical system 300 for implementing the embodiments of the method of FIGS. 5-8. The surgical system 300 includes a surgical robot 302, a computer system 304, and a tracking system 306. The surgical robot 302 may include a movable base 308, and has a manipulator arm 310 connected to the base 308, an end-effector 311 located at a distal end 312 of the manipulator arm 310, and a force sensor 314 positioned proximal to the end-effector 311 for sensing forces experienced on the end-effector 311. In some embodiments of the invention, the end-effector 311 is a drill for forming cavities, planes, or tunnels in bone for fixation of ligaments and tendons, as well as joint replacement implants. The base 308 may include a set of wheels 317 to maneuver the base 308, which may be fixed into position using a braking mechanism such as a hydraulic brake. The base 308 may further include an actuator to adjust the height of the manipulator arm 310. The manipulator arm 310 includes various joints and links to manipulate the end-effector 311 in various degrees of freedom. The joints are illustratively prismatic, revolute, spherical, or a combination thereof. The surgical robot 302 further includes a tracking reference device 420d to permit the tracking system 306 to track the position and orientation of the end-effector 311.

The computing system 304 generally includes an optional planning computer 316; a device computer 318; a tracking computer 320; and peripheral devices. The planning computer 316, device computer 318, and tracking computer 320 may be separate entities, one-in-the-same, or combinations thereof depending on the surgical system. It is appreciated that the planning computer 316; a device computer 318; a tracking computer 320 can be a unified computer that includes all such computers 316, 318, and 320; or a subset thereof. The location of computers, whether separate or unified is immaterial and each independently is located outside the operating room, within the operating room, or associated with the surgical robot 302. Further, in some embodiments, any combination of the planning computer 316, the device computer 318, and/or tracking computer 320 are connected via a wired or wireless communication. The peripheral devices allow a user to interface with the surgical system components and may include: one or more user-interfaces, such as a display or monitor 412 for the graphical user interface (GUI); and user-input mechanisms, such as a keyboard 414, mouse 422, pendant 424, joystick 426, foot pedal 428, or the monitor 412 that in some inventive embodiments has touchscreen capabilities.

The planning computer 316 is optional in that the methods described herein (e.g., the methods of FIGS. 5-8) may be performed without pre-operative or intra-operative planning using software operating on a computer to collect and manipulate data according to the inventive methods. However, in some instances, a surgeon may choose to review pre-operative images prior to the procedure to gauge or plan the location for operations on one or more bones. Therefore, the optional planning computer 316 may contain hardware (e.g., processors, controllers, and/or memory), software, data and utilities that are in some inventive embodiments dedicated to the review of any pre-operative or intra-operative images and to plan the operations on the subject bones and joints. This may include reading medical imaging data, segmenting imaging data, constructing three-dimensional (3D) virtual models, storing computer-aided design (CAD) files, providing various functions or widgets to aid a user in planning the surgical procedure, and generating surgical plan data. The final surgical plan may include image bone data, patient data, ligature implant and tunnel position data, trajectory parameters, and/or operational data.

It is appreciated that the force topography and point mapping on a bone may be visually represented on display or monitor 412 as a color spectrum where different colors along a continuum represent force magnitudes. It is also appreciated that the force topography may be represented in a grayscale, where for example a darker shade is representative of a higher applied force which equates to a higher density of the CT and of hardness of the layer. It is further appreciated that a series of gradient lines where a denser grouping of lines is representative of a harder region may also be used as an indicator of applied forces for corresponding layers.

The surgical plan data generated from the planning computer 316 may be displayed during the surgical procedure to assist the surgeon. If the planning computer 316 is located outside the OR, the surgical plan data may be transferred to the device computer 318, tracking computer 320, or other computer in communication with an OR display by way of a non-transient data storage medium (e.g., a compact disc (CD), a portable universal serial bus (USB) drive).

The device computer 318 in some inventive embodiments is housed in the moveable base 308 and contains hardware, software, data and utilities that are preferably dedicated to the operation of the surgical robotic device 302. This may include end-effector control, robotic manipulator control, the processing of kinematic and inverse kinematic data, the execution of calibration routines, the execution of operational data (e.g., trajectory parameters, guidance control), coordinate transformation processing, providing workflow instructions to a user, and utilizing position and orientation (POSE) data from the tracking system 306. In particular inventive embodiments, the device computer 318 records the entry point and exit point designated by the digitizer and calculates the vector between the entry point and exit point.

The surgical system 300 further includes a tracked digitizer probe 430 having a probe tip to determine the POSE of bones as described herein. The tracked digitizer probe 430 includes a tracking reference device 420c to permit the tracking system 306 to track the position and orientation of the probe 430 and the probe tip.

The tracking system 306 may be an optical tracking system that includes two or more optical receivers 307 to detect the position of fiducial markers (e.g., retroreflective spheres, active light emitting diodes (LEDs)) uniquely arranged on rigid bodies. The fiducial markers arranged on a rigid body are collectively referred to as a tracking array (420a, 420b, 420c, 420d), where each tracking array has a unique arrangement of fiducial markers, or a unique transmitting wavelength/frequency if the markers are active LEDs. An example of an optical tracking system is described in U.S. Pat. No. 6,061,644. The tracking system 306 may be built into a surgical light, located on a boom, a stand 334, or built into the walls or ceilings of the OR. The tracking system computer 320 may include tracking hardware, software, data, and utilities to determine the POSE of objects (e.g., bones B (Fibia-F, Tibia-T, surgical device 302) in a local or global coordinate frame. The POSE of the objects is collectively referred to herein as POSE data or tracking, where this POSE data may be communicated to the device computer 318 through a wired or wireless connection. The wireless communication may be accomplished via optical communication. Alternatively, the device computer 318 may determine the POSE data using the position of the fiducial markers detected from the optical receivers 307 directly.

The POSE data is determined using the position data detected from the optical receivers 307 and operations/processes such as image processing, image filtering, triangulation algorithms, geometric relationship processing, registration algorithms, calibration algorithms, and coordinate transformation processing.

The POSE data is used by the computing system 304 during the procedure to update the POSE and/or coordinate transforms of the vector (or entry and exit points) and the surgical robot 302 as the manipulator arm 310 and/or bone(s) (F, T) move during the procedure, such that the surgical robot 302 can accurately drill and perform operations in the designated locations on a bone.

The tracking system computer 320 may further record the location of the digitizer probe 430. The optical tracking system 306 may then send informational data, tracking data, and/or operational data to the device computer 318 to control or assist in the control of the end-effector 311 in performing operations on designated locations of a subject bone.

### OTHER EMBODIMENTS

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A method for determining the position and orientation of a bone of a patient comprising steps of:
directing a digitizer tip onto tissue overlying the bone to collect a first surface point location;
monitoring forces exerted on the digitizer tip while directing the digitizer;
determining when a force exerted on the digitizer tip exceeds a predetermined threshold force;
removing the digitizer tip to reduce the force exerted on the digitizer tip; and
recording a first registered location of the digitizer tip at an instant when the force is equal to, or less than the threshold force and indicative of the position and orientation of the bone.

2. The method of claim 1 further comprising repeating the aforementioned steps with the digitizer tip contacting at a plurality of surface point locations displaced from the first surface point to record a plurality of registered locations.

3. The method of claim 1 wherein at least one of said plurality of surface point locations is an exposed portion of the bone.

4. The method of claim 1 wherein the threshold force is based on premeasured bone hardness data obtained from a computed tomography (CT) scan of the bone or associated with the digitizer tip in direct contact with cortical bone.

5. The method of claim 1 further comprising repeating the aforementioned steps on a second bone that together with the bone define a joint or a portion of the joint.

6. The method of claim 1 further comprising using imaging data from a CT scan of the bone to determine material properties of the bone for a plurality of regions of the bone and determining a set of threshold forces for each of the plurality of bone regions based on the material properties in the plurality of bone regions.

7. The method of claim 1 wherein the threshold force is normalized to CT scan data.

8. The method of claim 1 wherein the digitizer tip contact with the first surface point location on the bone occurs during a surgical procedure.

9. A method for determining the position and orientation of a bone comprising steps of:
obtaining anatomy imaging data, where the anatomy imaging data includes voxels, where each voxel has an estimated tissue density value;
correlating the estimated tissue density values to an expected force value, where the expected force value is an expected measurement of force on a digitizer tip while digitizing a surface point;
directing a digitizer tip onto tissue overlying the bone to collect a first surface point location;
monitoring forces exerted on the digitizer tip while directing the digitizer;
recording the forces on the digitizer tip and the position of the digitizer tip while digitizing the first surface point location to generate a series of points, where each point in the series of points has a positional depth with an associated recorded force value or range;
correlating two or more points in the series of points with corresponding points in the anatomy imaging data based at least partially on a similarity between the expected force values and the recorded force values, where each point correlation represents a layer of tissue;
calculating at least one transformation matrix for a tissue layer using point correlation from at least some of the surface points digitized for the tissue layer; and
combining the at least one transformation matrix with additional matrix data to complete registration of the bone that is indicative of the position and orientation of the bone.

10. The method of claim 9 further comprising repeatedly directing the digitizer tip to a plurality of different surface locations relative to the first surface point location on the bone to record additional surface points.

11. The method of claim 9 wherein the transformation matrix is calculated using an iterative closest point (ICP) algorithm.

12. The method of claim 9 further comprising applying noise reduction and variability reduction to the registration.

13. A method for determining the position and orientation of a bone of a patient comprising:
obtaining imaging data from a CT scan of the bone;
directing a digitizer tip onto tissue overlying the bone to collect a first surface point location;
monitoring forces exerted on the digitizer tip while directing the digitizer tip;
recording the forces on the digitizer tip and the position of the digitizer tip while digitizing the first surface point location to generate a plurality of points, where each point in the plurality of points has a positional depth with an associated recorded force value or range of values;
correlating two or more points in the plurality of points with corresponding points in the anatomy imaging data based at least partially on a similarity between an expected force threshold and the recorded force values, where each point correlation represents a layer of tissue;
calculating at least one transformation matrix for a tissue layer using each point correlation from at least some of the surface points digitized for the tissue layer; and
combining the at least one transformation matrix with additional matrix data to complete registration of the bone that is indicative of the position and orientation of the bone.

14. The method of claim 13 further comprising repeatedly directing the digitizer tip to different locations on the bone to collect additional surface point locations.

15. The method of claim 13 wherein the at least one transformation matrix is calculated using iterative closest point (ICP) algorithms.

16. The method of claim 13 further comprising applying noise reduction and variability reduction to the registration.

17. A computer-assisted surgical system, comprising:
a percutaneous bone detector having a digitizer tip to collect a set of surface point locations on a bone of a patient;
a tracking system;
a surgical robot with an end effector;
one or more computers with software, wherein said one or more computers receive signals from the tracking system which tracks the position of the digitizer tip and records forces exerted on the digitizer tip;
a display to display the output from the one or more computers; and
wherein the one more computers with software execute instructions to perform at least one of the following: (a) record a position of the digitizer tip when a force on the digitizer tip in contact with the bone is equal to or less than a threshold force; or (b) calculate at least one transformation matrix representative of a tissue layers and combine the at least one transformation matrix with additional matrix data to register the bone.

18. The system of claim 17 wherein the percutaneous bone detector further comprises a load cell that detects the forces exerted on the digitizer tip.

19. The system of claim 18 wherein the load cell is a strain gauge.

20. The system of claim 17 wherein the tracking system is at least one of a mechanical arm having the bone detector assembled thereto, or an optical tracking system.
